(19) United States
(12) Patent Application Publication
(10) Pub. No.: US 2011/0152658 A1
(43) Pub. Date: Jun. 23, 2011

(54) IDENTIFICATION OF ABERRANT MEASUREMENTS OF IN VIVO GLUCOSE CONCENTRATION USING TEMPERATURE

(75) Inventors: Thomas A. Peyser, Menlo Park, CA (US); Richard E. Purvis, Pasadena, CA (US); David R. Markle, Berwyn, PA (US); Soya Gamsey, Huntington Beach, CA (US); Matthew A. Romey, Newport Beach, CA (US)

(73) Assignee: GLUMETRICS, INC., Irvine, CA (US)

(21) Appl. No.: 12/972,385

(22) Filed: Dec. 17, 2010

Related U.S. Application Data
(60) Provisional application No. 61/287,656, filed on Dec. 17, 2009.

(51) Int. Cl. A61B 5/145

(52) U.S. Cl. 600/365

(57) ABSTRACT

Disclosed herein are methods and systems for generating an estimate of an in vivo analyte concentration and identifying whether the estimate is aberrant. In some embodiments, the system includes a sensor comprising an analyte sensor and a temperature sensing element, and a control unit programmed to identify changes in temperature that may indicate a non-physiologic condition (and result in an aberrant glucose measurement). In some embodiments, the methods include generating an estimate of analyte concentration at a particular time using the analyte sensor, and generating first and second signals indicative of temperature using the temperature sensing element. In some embodiments the methods include identifying the estimate of analyte concentration as aberrant if the magnitude of the difference between the first and second signals indicative of temperature exceeds a threshold value.

### CROSS-REFERENCE TO RELATED APPLICATIONS

This application claims priority to U.S. Provisional Patent Application No. 61/287,656, filed Dec. 17, 2009, the disclosure of which is hereby expressly incorporated by reference and hereby expressly made a portion of this application. This application is also related to co-pending U.S. patent application Ser. Nos. 11/671,880, filed on Feb. 6, 2007, now U.S. Pat. No. 7,751,863 issued on Jul. 6, 2010; 12/027,158, filed on Feb. 6, 2008; 12/026,396, filed on Feb. 5, 2008; 12/118,429, filed on May 9, 2008; 12/118,401, filed on May 9, 2008; 12/274,617, filed on Nov. 20, 2008; and 12/424,902, filed on Apr. 16, 2009; the disclosure of each of which is hereby expressly incorporated by reference in its entirety and is hereby expressly made a portion of this application.

### BACKGROUND OF THE INVENTION

1. Field of the Invention

Disclosed herein are systems and methods relating to in vivo measurements of analyte concentration and the use of temperature to interpret or adjust these measurements, or identify the measurements as aberrant.

1. Description of the Related Art

Hyperglycemia and insulin resistance are common in critically ill patients, even if such patients have not previously had diabetes. In these situations, glucose levels rise in critically ill patients thereby increasing the risk of damage to a patient's organs. Further, studies have shown that normalization of blood glucose levels with insulin therapy improves the prognosis for such patients, thereby decreasing mortality rates.

More recent scientific evidence confirms that dramatic improvements in the clinical outcome of hospitalized Intensive Care Unit (ICU) patients can result from therapeutic control of blood glucose to normal ranges. These studies indicate that glycemic control (GC) of ICU patients may reduce mortality by as much as 40%, and significantly lower complication rates. In these situations, it is desirable to accurately, conveniently and substantially continuously monitor blood sugar in a real-time or near real-time device specifically designed to meet the challenging needs of the ICU environment. Researchers at Johns Hopkins University estimate that GC can save as many as 150,000 lives and reduce U.S. healthcare costs by as much as $18 billion annually.

Performing GC is facilitated by continuous, nearly continuous, or intermittent monitoring of blood glucose levels. One factor that can affect the accuracy of a blood glucose determination is the temperature of the sensor when the determination is made, especially if the sensor output is highly sensitive to temperature. Such changes in temperature can result from a change in the temperature of the patient being monitored as well as the location of the sensor within the patient (such as in the arm versus the body core or another part of the body). Using a temperature sensing element to correct glucose readings for temperature effects is known. See e.g., W02008/001091 to Crane, et al. and U.S. Patent Publication No. 2005/0056539 to Morgan, et al.; each of which is incorporated herein in its entirety by reference thereto.

In addition, when glucose is being monitored in a hospital setting, it is common for the patient to be administered intravenous (IV) and/or arterial fluids. Where the glucose sensor is deployed intravascularly in close proximity and/or just downstream to the infusion port, or deployed within the same vascular line used for infusion of fluids and downstream to the infusion port, then the resultant glucose reading may be aberrant—reflecting the contributions of the IV fluid. If an aberrant glucose measurement is used for some medical decisions or for control of blood glucose level, such as by administration of insulin, an improper dose could be given or an incorrect and potentially hazardous medical decision could be made. Consequently, monitoring systems and methods are needed for alerting and/or otherwise preventing hospital staff from relying on aberrant glucose readings based on non-representative sensor environment conditions.

### SUMMARY OF THE INVENTION

Disclosed herein are systems for measuring an in vivo glucose concentration and identifying whether the measurement is aberrant. In some embodiments, the system has a glucose sensor with a chemical indicator system that generates a signal related to glucose concentration, and a temperature sensor that measures the temperature of or nearby the fluid being measured by the glucose sensor. In some embodiments, the system also has one or more sensor control unit that can control the operation of the glucose sensor and the temperature sensor. The system can also have one or more receiving and processing unit that receives the glucose signal and temperature signal during the operation of the system. The system can also be configured to compare the temperature signal of one point to another, the other being earlier or later in time. The system can also be configured to generate a metric indicative of the difference in temperature at the glucose sensor between the first time and second time, which can be the temperature signals immediately after each other, or from a fixed set point to some later point or earlier point.

Also disclosed herein are methods of generating an estimate of an in vivo analyte concentration and identifying whether the estimate is aberrant. In some embodiments, the methods include providing in vivo a sensor comprising an analyte sensor and a temperature sensing element. In some embodiments, the methods include generating an estimate of analyte concentration at a particular time using the analyte sensor. In some embodiments, the methods include generating a first signal indicative of temperature using the temperature sensing element and generating a second signal indicative of temperature at the particular time using the temperature sensing element. In some embodiments, the methods include identifying the estimate of analyte concentration as aberrant if the magnitude of the difference between the first signal indicative of temperature and the second signal indicative of temperature exceeds a threshold value. In some embodiments, the methods include computing a metric from the first signal indicative of temperature and the second signal indicative of temperature, and identifying the estimate of analyte concentration as aberrant if the metric exceeds a threshold value.

Also disclosed herein are methods of identifying whether a measured analyte concentration, measured by an in vivo analyte sensor at a particular time, is aberrant. In some embodiments, the methods include generating a first signal indicative of a temperature at the analyte sensor previous in time to the particular time, and generating a second signal indicative of a temperature at the analyte sensor at the particular time. In some embodiments, the methods include generating a metric from the first signal and the second signal, the metric indicative of a difference in temperature at the analyte sensor between the particular time and a previous time. In some embodiments, the methods include identifying the measured analyte concentration as aberrant if the metric exceeds a threshold value. In some embodiments, the methods further include alerting a user, alerting a monitor, and/or signaling a medication delivery device when an aberrant analyte measurement is identified.

Also disclosed herein are methods of administering medication to a patient in response to a measured concentration of an analyte in the patient. In some embodiments, the methods include providing in vivo an analyte sensor, providing in vivo a temperature sensing element. In some embodiments, the methods include measuring the concentration of the analyte using the analyte sensor, and identifying whether the measured concentration of the analyte is aberrant according to a method described herein using the temperature sensing element. In some embodiments, the methods include administering medication to the patient in response to the measured concentration of the analyte if and only if the measured concentration is not aberrant. In some embodiments, the analyte is glucose.

Also disclosed herein are systems for measuring an in vivo concentration of an analyte at a particular time and identifying whether the measurement is aberrant. In some embodiments, the system includes an analyte sensor configured to generate a signal indicative of the in vivo concentration of the analyte at the particular time. In some embodiments, the system includes a temperature sensing element configured to generate a plurality of signals including a first signal indicative of temperature, and a second signal indicative of temperature at the particular time. In some embodiments, the system includes at least one sensor control unit which is configured to control the operation of the analyte sensor and the temperature sensing element. In some embodiments, the system includes at least one receiving and processing unit. In some embodiments, the receiving and processing unit is configured to receive the signal indicative of the in vivo concentration of the analyte, receive the first signal indicative of temperature, and receive the second signal indicative of temperature. In some embodiments, the receiving and processing unit is configured to generate a metric from the first signal indicative of temperature and the second signal indicative of temperature. In some embodiments, the metric generated by the receiving and processing unit is indicative of the difference in temperature at the analyte sensor between the particular time and a previous time. In some embodiments, the receiving and processing unit identifies the analyte concentration as aberrant if the metric exceeds a threshold value. In some embodiments, the analyte is glucose.

### BRIEF DESCRIPTION OF THE DRAWINGS

FIG. 1 is a cut-away view of a sensor where a portion of the porous membrane sheath is cut away to expose the optical fiber and hydrogel beneath the membrane.

FIG. 2 is a cross-sectional view along a longitudinal axis of a sensor with a hydrogel disposed distal the optical fiber.

FIG. 3A shows a glucose sensor having a series of holes that form a helical configuration.

FIG. 3B shows a glucose sensor having a series of holes drilled or formed at an angle.

FIG. 3C shows a glucose sensor having at least one spiral groove.

FIG. 3D shows a glucose sensor having a series of triangular wedge cut-outs.

FIG. 4 shows a cross-sectional view of one embodiment of a glucose sensor having a cavity in the distal portion of the sensor.

FIG. 5 shows a glucose measurement system comprising two excitation light sources and a microspectrometer and/or spectrometer.

FIG. 6 is a block diagram showing the components and connectivity of an analyte monitoring system in accordance with an embodiment of the present invention.

### DETAILED DESCRIPTION OF THE PREFERRED EMBODIMENT

The following description and examples illustrate a preferred embodiment of the present invention in detail. Those of skill in the art will recognize that there are numerous variations and modifications of this invention that are encompassed within its scope. Accordingly, the description of a preferred embodiment should not be deemed to limit the scope of the present invention.

Embodiments of the present invention relate to measuring the activity of a particular analyte (e.g., glucose, potassium, etc.) in a physiologic fluid, e.g., blood or interstitial fluid, using a sensor configured to measure the amount of free, bioavailable analyte dissolved in the water compartment of the physiologic fluid, without significantly perturbing the equilibrium between free analyte in the water compartment and analyte that is otherwise bound or associated with molecules or cells. The phrases free and bioavailable analyte, or analyte activity, are used generally herein to refer to the amount of analyte (preferably expressed in mmoles) per unit of water (preferably expressed in kg). This measure of analyte activity focuses on the physiologically relevant amount of analyte (as opposed to the total concentration of analyte in the fluid/suspension). Preferably, analyte activity measurements minimize or exclude contributions from analyte that is not freely dissolved and bioavailable (such as e.g., analyte that may be aggregated, complexed with other molecules, bound to receptors, or associated with macromolecules, proteins, glycoproteins, lipids, glycolipids, etc., or sequestered within cells and organelles, etc.). In accordance with embodiments of the invention, the measured analyte activity is then utilized to adjust or maintain the physiologic analyte activity at a desired level, for example, by interfacing manually or automatically with means for raising or lowering the amount of analyte activity. Note, that in the description that follows, the term “concentration” is often used to refer to the quantity of analyte in solution. It is to be understood, however, that the term “concentration” also refers to the “activity” of the analyte in solution, as explained in this paragraph.

One particular analyte of interest is glucose. Maintaining proper levels of bioavailability glucose has been found critical to the successful recovery of bedridden patients. The concentration of bioavailability glucose in a patient's whole blood may be referred to as a patient's glucose activity. Monitoring and maintaining a patient's glucose activity is an ongoing problem for hospital staff. Accordingly, disclosed herein are systems and methods which may automatically (or somewhat automatically) regulate a patient's glucose activity.

Therefore, among the embodiments disclosed herein are various glucose monitoring systems comprising intravascular glucose sensors which further comprise temperature sensing element(s). Also among the embodiments disclosed herein are methods for alerting hospital staff and preventing pharmacologic intervention based on glucose readings that may be aberrant, e.g., due to infusion of IV fluids in close proximity to the sensor. Of course, intravascular sensors for detecting and determining the activity of other analytes besides glucose may also benefit from aspects of the invention, e.g., using a temperature sensing element to detect changes in temperature at or near the analyte sensor that may give rise to aberrant readings. Other analytes for which the intravascular activity level may be measured include for example, oxygen, carbon dioxide, lactate, calcium, sodium, magnesium, potassium, carbon monoxide, etc.

### Systems and Methods Overview

Analyte monitoring systems in accordance with embodiments of the present invention may include: an analyte (preferably glucose) sensor configured for intravascular deployment and comprising a temperature sensing element; and a sensor control unit operably coupled to the analyte sensor and including, for example, means for providing energy to the sensor (e.g., excitation light or voltage) and means for receiving and processing signals from the sensor. The sensor control unit may evaluate the signals from the sensor and/or transmit the signals to one or more optional receiver/display units for evaluation. The sensor control unit and/or the receiver/display units may display or otherwise communicate the current level of analyte. Furthermore, the sensor control unit and/or the receiver/display units may indicate to the patient and/or hospital staff, via, for example, an audible, visual, or other sensory-stimulating alarm, when the analyte reading is at or near a threshold level, trending toward a threshold level, and/or may be aberrant due to non-representative sensor environment conditions.

Where the sensor is deployed intravascularly in close proximity and/or just downstream to an intravenous or intraarterial infusion port, or deployed within the same vascular line used for infusion of fluids and downstream to the infusion port, then the resultant analyte reading may be aberrant—reflecting the contributions of the infusion fluids. The infused fluid may be high or low in analyte concentration (compared to blood) and/or may dilute the blood in the vicinity of the sensor. The infusion fluid is also likely to change the temperature (e.g., decrease the temperature) of the blood at or just downstream to the infusion port. Accordingly, the detection of a change in temperature of the blood in contact with the sensor can be used, in accordance with preferred embodiments, to signal the sensor control unit and/or the receiver/display units that contemporaneous analyte readings may be suspect, aberrant and/or reflect non-representative conditions.

In some embodiments, a glucose monitoring system may be operably coupled to a drug delivery device adapted to automatically administer a glucose modulating agent, e.g., insulin, dextrose, etc, in response to a glucose reading that is at or near a threshold level and/or trending toward a threshold level. In such automated systems for maintaining tight glycemic control, it is preferred that the glucose modulating agents are not delivered based on an aberrant reading from the sensor. Thus, as described above, the temperature sensing element can be used to detect changes in the temperature of the blood in contact with the sensor, and thereby prevent automatic administration of a glucose modulating agent in response to a glucose reading that may be suspect, aberrant and/or non-representative due e.g., to the presence of infusion fluids.

### Glucose Sensors

The glucose monitoring systems of the present invention can be utilized under a variety of conditions. The particular configuration of a sensor and other units used in the glucose monitoring system may depend on the use for which the glucose monitoring system is intended and the conditions under which the glucose monitoring system will operate. One embodiment of the glucose monitoring system includes a sensor configured for implantation into a patient. For example, implantation of the sensor may be made in the arterial or venous systems for direct testing of glucose levels in blood. The site of implantation may affect the particular shape, components, and configuration of the sensor. Examples of glucose sensors configured for intravascular deployment include the optical sensors disclosed in U.S. Pat. Nos. 5,137,033, 5,512,246, 5,503,770, 6,627,177, 7,417,164 and 7,470,420, 7,824,918, 7,829,341, and U.S. Patent Publ. Nos. 2008/0188722, 2008/0188725, 2008/0187655, 2008/0305009, 2009/0018426, 2009/0018418, 2009/0177143, and 2009/0264719; each of which is incorporated herein in its entirety by reference thereto.

Other glucose sensors configured for intravascular deployment include electrochemical sensors, such as those disclosed in U.S. Patent Publ. Nos. 2008/0119704, 2008/0197024, 2008/0200788, 2008/0200789 and 2008/0200791; each of which is incorporated herein in its entirety by reference thereto.

An optical glucose sensor in accordance with preferred embodiments of the present invention comprises a chemical indicator system. Some useful indicator systems comprise a fluorophore operably coupled to an analyte binding moiety, wherein analyte binding causes an apparent optical change in the fluorophore concentration (e.g., emission intensity). For example, a glucose binding moiety such as 3,3′-oBBV that is operably coupled to a fluorescent dye such as HPTS-triCysMA will quench the emission intensity of the fluorescent dye, wherein the extent of quenching is reduced upon glucose binding resulting in an increase in emission intensity related to glucose concentration. In further preferred embodiments, the indicator systems also comprise a means for immobilizing the sensing moieties (e.g., dye-quencher) such that they remain physically close enough to one another to react (quenching). Such immobilizing means are preferably insoluble in an aqueous environment (e.g., intravascular), permeable to the target analytes, and impermeable to the sensing moieties. Typically, the immobilizing means comprises a water-insoluble organic polymer matrix. For example, the HPTS-triCysMA dye and 3,3′-oBBV quencher may be effectively immobilized within a DMAA (N,N-dimethylacrylamide) hydrogel matrix.

Some preferred fluorophores (e.g., HPTS-triCysMA), quenchers/analyte binding moieties (e.g., 3,3′ -oBBV) and immobilizing means (e.g., N,N-dimethylacrylamide), as well as methods for synthesizing and assembling such indicator systems are set forth in greater detail in U.S. Pat. Nos. 6,627,177, 7,417,164 7,470,420, 7,829,341 and U.S. Patent Publ. Nos. 2008/0188722, 2008/0188725, 2008/0187655, 2008/0305009, 2009/0018426, 2009/0018418, 2009/0061528, 2009/0177143, and 2009/0264719.

Other indicator chemistries, such as those disclosed in U.S. Pat. Nos. 5,176,882 to Gray et al. and 5,137,833 to Russell, can also be used in accordance with embodiments of the present invention; both of which are incorporated herein in their entireties by reference thereto.

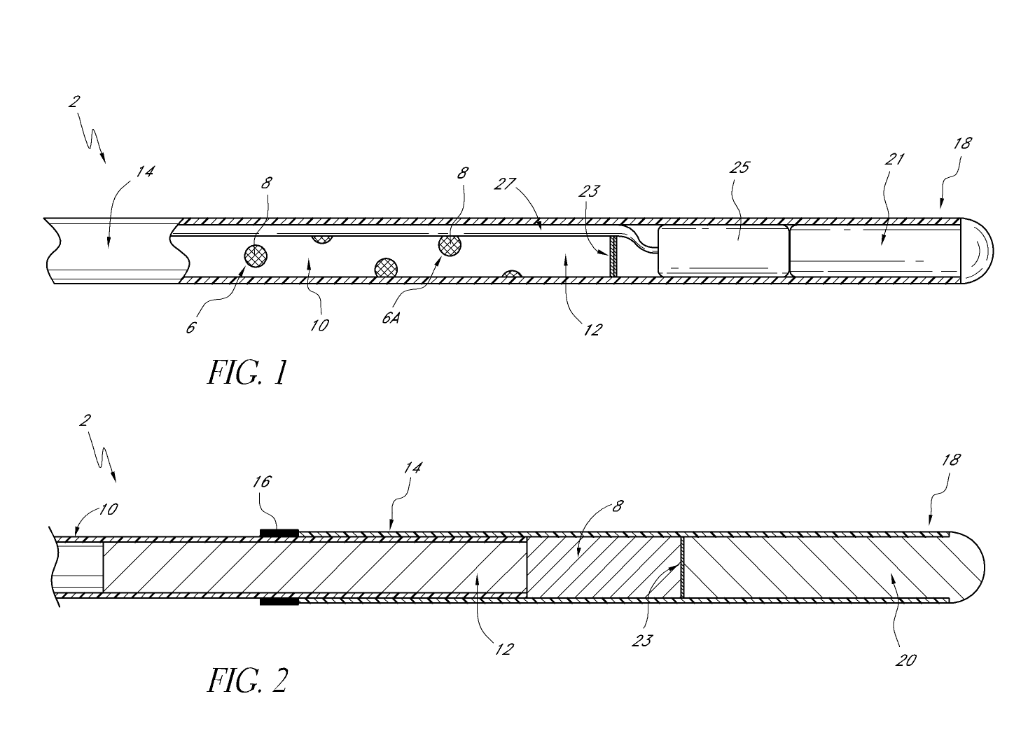

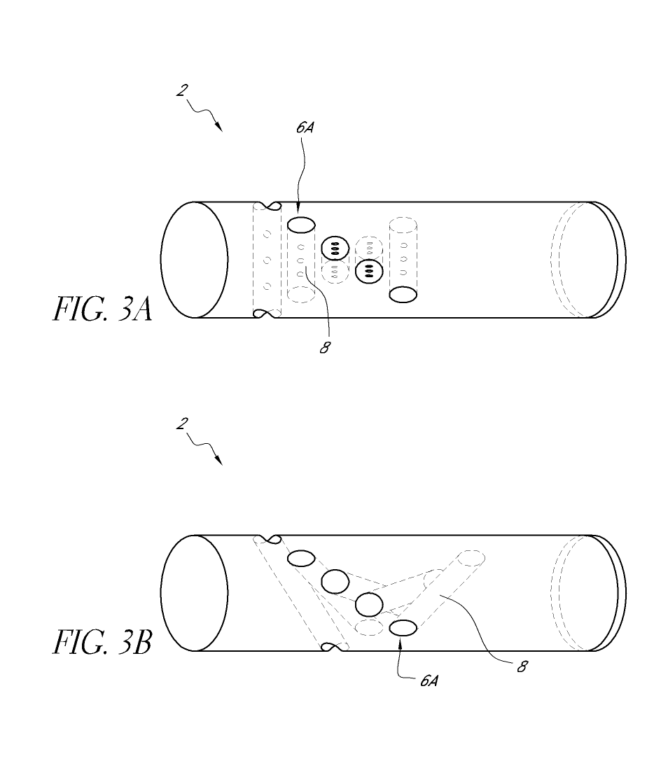

FIG. 1 shows a sensor 2 in accordance with an embodiment of the present invention. The sensor comprises an optical fiber 10 with a distal end 12 disposed in a porous membrane sheath 14. The optical fiber 10 has cavities, such as holes 6A, in the fiber optic wall that can be formed by, for example, mechanical means such as drilling or cutting. The holes 6A in the optical fiber 10 can be filled with a suitable compound, such as a polymer. In some embodiments, the polymer is a hydrogel 8. In other embodiments of the sensor 2 as shown in FIG. 2, the optical fiber 10 does not have holes 6A, and instead, the hydrogel 8 is disposed in a space distal to the distal end 12 of the optical fiber 10 and proximal to the mirror 23. In some embodiments, the sensor 2 is a glucose sensor. In some embodiments, the glucose sensor is an intravascular glucose sensor.

In some embodiments, the porous membrane sheath 14 can be made from a polymeric material such as polyethylene, polycarbonate, polysulfone or polypropylene. Other materials can also be used to make the porous membrane sheath 14 such as zeolites, ceramics, metals, or combinations of these materials. In some embodiments, the porous membrane sheath 14 is microporous and has a mean pore size that is less than approximately two nanometers. In other embodiments, the porous membrane sheath 14 is mesoporous and has a mean pore size that is between approximately two nanometers to approximately fifty nanometers. In still other embodiments, the porous membrane sheath 14 is macroporous and has a mean pore size that is greater than approximately fifty nanometers.

In some embodiments as shown in FIG. 2, the porous membrane sheath 14 is attached to the optical fiber 10 by a connector 16. For example, the connector 16 can be an elastic collar that holds the porous membrane sheath 14 in place by exerting a compressive force on the optical fiber 10, as shown in FIG. 2. In other embodiments, the connector 16 is an adhesive or a thermal weld.

In some embodiments as shown in FIG. 1, a mirror 23 and thermistor 25 can be placed within the porous membrane sheath 14 distal the distal end 12 of the optical fiber 10. Thermistor leads 27 can be made to run in a space between the optical fiber 10 and porous membrane sheath 14. Although a thermistor 25 is shown, other devices such as a thermocouple, pressure transducer, an oxygen sensor, a carbon dioxide sensor or a pH sensor for example can be used instead.

In some embodiments as shown in FIG. 2, the distal end 18 of the porous membrane sheath 14 is open and can be sealed with, for example, an adhesive 20. In some embodiments, the adhesive 20 can comprise a polymerizable material that can fill the distal end 18 and then be polymerized into a plug. Alternatively, in other embodiments the distal end 18 can be thermally welded by melting a portion of the polymeric material on the distal end 18, closing the opening and allowing the melted polymeric material to resolidify. In other embodiments as shown in FIG. 1, a polymeric plug 21 can be inserted into the distal end 18 and thermally heated to weld the plug to the porous membrane sheath 14. Thermoplastic polymeric materials such as polyethylene, polypropylene, polycarbonate and polysulfone are particularly suited for thermal welding. In other embodiments, the distal end 18 of the porous membrane sheath 14 can be sealed against the optical fiber 10.

After the porous membrane sheath 14 is attached to the optical fiber 10 and the distal end 18 of the porous membrane sheath 14 is sealed, the sensor 2 can be vacuum filled with a first solution comprising a monomer, a crosslinker and a first initiator. Vacuum filling of a polymerizable solution through a porous membrane and into a cavity in a sensor is described in detail in U.S. Pat. No. 5,618,587 to Markle et al.; incorporated herein in its entirety by reference thereto. The first solution is allowed to fill the cavity 6 within the optical fiber 10.

In some embodiments, the first solution is aqueous and the monomer, the crosslinker and the first initiator are soluble in water. For example, in some embodiments, the monomer is acrylamide, the crosslinker is bisacrylamide and the first initiator is ammonium persulfate. In other embodiments, the monomer is dimethylacrylamide or N-hydroxymethylacrylamide. By increasing the concentrations of the monomer and/or crosslinker, the porosity of the resulting gel can be decreased. Conversely, by decreasing the concentrations of the monomer and/or crosslinker, the porosity of the resulting gel can be increased. Other types of monomers and crosslinkers are also contemplated. In other embodiments, the first solution further comprises an analyte indicator system comprising a fluorophore and an analyte binding moiety that functions to quench the fluorescent emission of the fluorophore by an amount related to the concentration of the analyte. In some embodiments, the fluorophore and analyte binding moiety are immobilized during polymerization, such that the fluorophore and analyte binding moiety are operably coupled. In other embodiments, the fluorophore and analyte binding moiety are covalently linked. The indicator system chemistry may also be covalently linked to the polymeric matrix.

In some embodiments, after the sensor 2 is filled with the first solution, the optical fiber 10 and the first solution filled porous membrane sheath 14 and cavity are transferred to and immersed into a second solution comprising a second initiator. In some embodiments, the second solution is aqueous and the second initiator is tetramethylethylenediamine (TEMED). In some embodiments, the second solution further comprises the same fluorescent dye and/or quencher found in the first solution and in substantially the same concentrations. By having the fluorescent dye and quencher in both the first solution and the second solution, diffusion of fluorescent dye and quencher out of the first solution and into the second solution can be reduced. In some embodiments where a second solution is used, the second solution further comprises monomer in substantially the same concentration as in the first solution. This reduces diffusion of monomer out of the first solution by reducing the monomer gradient between the first solution and the second solution.

In some embodiments, at or approximately at the interface between the first and second solutions, the first initiator and the second initiator can react together to generate a radical. In some embodiments, the first initiator and the second initiator react together in a redox reaction. In other embodiments, the radical can be generated by thermal decomposition, photolytic initiation or initiation by ionizing radiation. In these other embodiments, the radical may be generated anywhere in the first solution. Once the radical is generated, the radical can then initiate polymerization of the monomer and crosslinker in the first solution.

When the radical is generated via a redox reaction as described herein, the polymerization proceeds generally from the interface between the first and second solutions to the interior of the porous membrane sheath 14 and towards the cavity in the optical fiber 10. Rapid initiation of polymerization can help reduce the amount of first initiator that can diffuse from the first solution and into the second solution. Reducing the amount of first initiator that diffuses out of the first solution helps reduce polymerization of monomer outside the porous membrane sheath 14 which helps in forming a smooth external surface. Polymerization of the monomer and crosslinker results in a hydrogel 8 that in some embodiments substantially immobilizes the indicator system, forming the sensor 2. Further variations on polymerization methodologies are disclosed in U.S. Patent Publ. No. 2008/0187655; incorporated herein in its entirety by reference thereto.

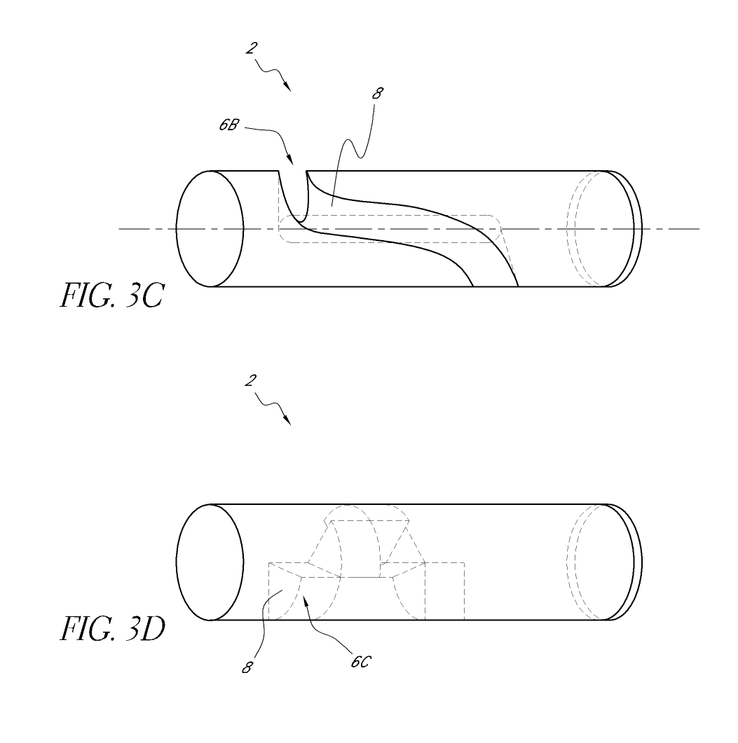

With reference to FIG. 3A, in certain embodiments, the glucose sensor 2 is a solid optical fiber with a series holes 6A drilled straight through the sides of the optical fiber. In certain embodiments, the holes 6A are filled with the hydrogels 8. In certain embodiments, the series of holes 6A that are drilled through the glucose sensor 2 are evenly spaced horizontally and evenly rotated around the sides of the glucose sensor 2 to form a spiral or helical configuration. In certain embodiments, the series of holes 6A are drilled through the diameter of the glucose sensor 2. With reference to FIG. 3B, in certain embodiments, the glucose sensor 2 is a solid optical fiber with a series of holes 6A drilled through the sides of the fiber at an angle. In certain embodiments, the series of holes 6A drilled at an angle, which are filled with hydrogel 8, are evenly spaced horizontally and evenly rotated around the sides the glucose sensor 2. With reference to FIG. 3C, in certain embodiments, the optical fiber comprises a groove 6B along the length of the optical fiber, wherein the groove 6B is filled with hydrogel 8. In certain embodiments, the depth of the groove 6B extends to the center of the optical fiber. In certain embodiments, the groove 6B spirals around the optical fiber. In certain embodiments, the groove 6B spirals around the optical fiber to complete at least one rotation. In certain embodiments, the groove 6B spirals around the optical fiber to complete multiple rotations around the optical fiber.

With reference to FIG. 3D, in certain embodiments, the glucose sensor 2 is a solid optical fiber with triangular wedges 6C cut from the fiber. In certain embodiments, the triangular wedge areas 6C are filled with hydrogel 8. In certain embodiments, the triangular wedges cut-outs 6C are evenly spaced horizontally and around the sides of the glucose sensor 2. In certain embodiments, all light traveling in the glucose sensor 2 is transmitted through at least one hole 6A or groove 6B filled with hydrogel.

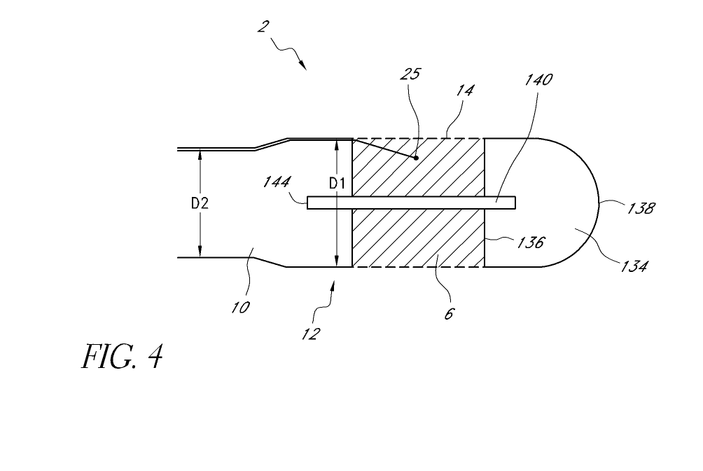

In certain embodiments, as illustrated in FIG. 4, the glucose sensor 2 comprises an optical fiber 10 having a distal end 12, an atraumatic tip portion 134 having a proximal end 136 and a distal end 138, a cavity 6 between the distal end 132 of the optical fiber 130 and the proximal end 136 of the atraumatic tip portion 134, and a rod 140 connecting the distal end 132 of the optical fiber 130 to the proximal end 136 of the atraumatic tip portion 134. A hydrogel 8 containing glucose sensing chemistry, for example a fluorophore and quencher, fills the cavity 6. Covering the hydrogel filled cavity 6 is a selectively permeable membrane 14 that allows passage of glucose into and out of the hydrogel 8. Although these embodiments are described using a glucose sensor 2, it should be understood by a person of ordinary skill in the art that the sensor 2 can be modified to measure other analytes by changing, for example, the sensing chemistry, and if necessary, the selectively permeable membrane 14. The proximal portion of the sensor 2 comprises the proximal portion 12 of the optical fiber 10. In some embodiments, the diameter, D1, of the distal portion of the sensor 2 is greater than the diameter, D2, of the proximal portion of the sensor 2. For example, the diameter D1 of the distal portion of the sensor 2 can be between about 0.0080 inches and 0.020 inches, while the diameter D2 of the proximal portion of the sensor 2 can be between about 0.005 inches to 0.015 inches. In some embodiments, the diameter D1 of the distal portion of the sensor 2 is about 0.012 inches, while the diameter D2 of the proximal portion of the sensor 2 is about 0.010 inches.

In some embodiments, the glucose sensor 2 includes a temperature sensor 25, such as thermocouple or thermistor. The temperature sensor 25 can measure the temperature of the hydrogel 8 and glucose sensing chemistry system. The temperature sensor 25 is particularly important when the glucose sensing chemistry, such as a fluorophore system, is affected by temperature change. For example, in some embodiments, the fluorescence intensity emitted by the fluorophore system is dependent on the temperature of the fluorophore system. By measuring the temperature of the fluorophore system, temperature induced variations in fluorophore fluorescence intensity can be accounted for, allowing for more accurate determination of glucose concentration, as more fully described below.

In certain embodiments, the hydrogels are associated with a plurality of fluorophore systems. In certain embodiments, the fluorophore systems comprise a quencher with a glucose receptor site. In certain embodiments, when there is no glucose present to bind with the glucose receptor, the quencher prevents the fluorophore system from emitting light when the dye is excited by an excitation light. In certain embodiments, when there is glucose present to bind with the glucose receptor, the quencher allows the fluorophore system to emit light when the dye is excited by an excitation light.

In certain embodiments, the emission produced by the fluorophore system varies with the pH of the solution (for example, blood), such that different excitation wavelengths (one exciting the acid form of the fluorophore and the other the base form of the fluorophore) produce different emission signals. In preferred embodiments, the ratio of the emission signal from the base form of the fluorophore over the emission signal from the acid form of the fluorophore is related to the pH level of the blood; the simultaneous measurement of glucose and pH is described in detail in U.S. Patent Publication No. 2008/0188722 (incorporated herein in its entirety by reference thereto). In certain embodiments, an interference filter is employed to ensure that the two excitation lights are exciting only one form (the acid form or the base form) of the fluorophore.

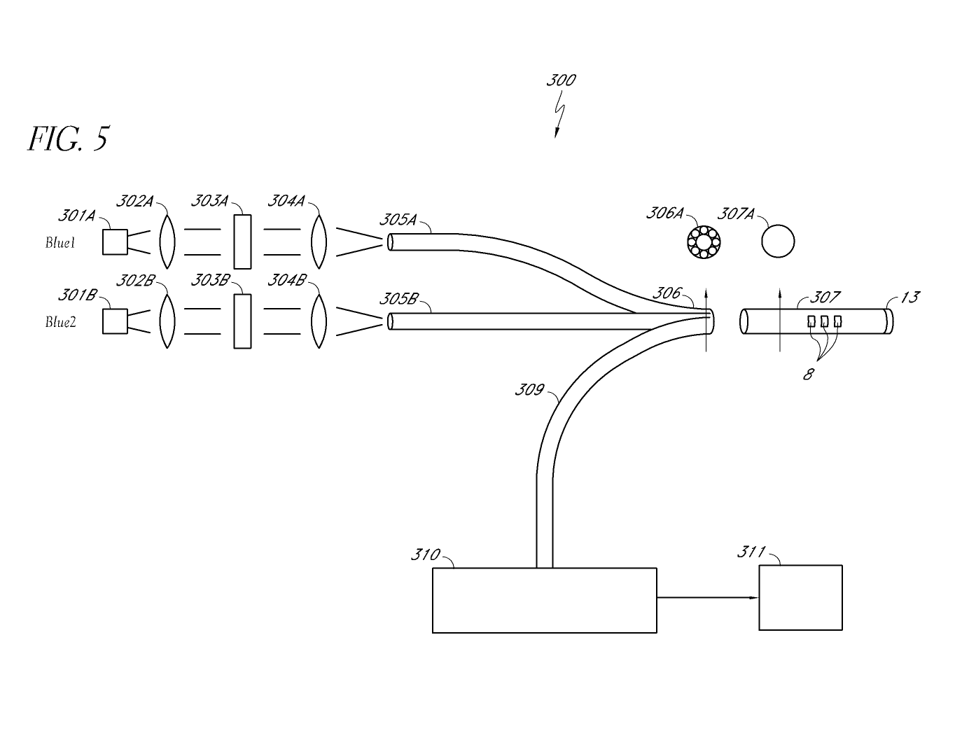

Variations in optical sensing systems, light sources, hardware, filters, and detection systems are described in detail in U.S. Publication No. 2008/0188725; incorporated herein in its entirety by reference thereto. See e.g., FIG. 5, wherein certain embodiments comprise at least two light sources. In certain embodiments, the light sources 301A, 301B generate excitation light that is transmitted through a collimator lens 302A, 302B. In certain embodiments, the resulting light from collimator lens 302A, 302B is transmitted to interference filters 303A, 303B. In certain embodiments, the resulting light from interference filters 303A, 303B is focused by focusing lens 304A, 304B into fiber optic lines 305A, 305B. In certain embodiments, fiber optic lines 305A, 305B may be a single fiber or a bundle of fibers. In certain embodiments, the fiber optic line 309 may be a single fiber or a bundle of fibers. In certain embodiments, fiber optic lines 305A, 305B, 309 are bundled together at junction 306 and are connected at glucose sensor 307. The glucose sensor 307 comprises hydrogels 8.

In certain embodiments, the emission light and the excitation light are reflected off the mirror 13 and into the fiber optic line 309. In certain embodiments, the fiber optic line 309 is connected to microspectrometer 310 that measures the entire spectrum of light in the glucose measurement system 300. The microspectrometer 310 may be coupled to a data processing module 311, e.g., the sensor control unit and/or receiver/display unit. In certain embodiments, the ratio of emission light over the corresponding excitation light is related to the concentration of glucose. In certain embodiments, the ratio of the emissions light (for example, the base form) produced by the first excitation light over the emission light (for example, the acid form) produced by the second excitation light is related to pH levels in the test solution, for example blood.

In certain preferred embodiments, the microspectrometer is the UV/VIS Microspectrometer Module manufactured by Boehringer Ingelheim. Any microspectrometer can be used. Alternatively, the microspectrometer could be substituted with other spectrometer, such as those manufactured by Ocean Optic Inc.

In certain embodiments described above, the ratiometric calculations require measurements of various light intensities. In certain embodiments, these measurements are determined by measuring the peak amplitudes at a particular wavelength or wavelength band. In certain embodiments, these measurements are determined by calculating the area under the curve between two particular wavelengths as for example with the output from a microspectrometer.

### Temperature Sensing Elements

As discussed above, a temperature sensing element, otherwise referred to herein as a temperature sensor or probe, is included in preferred embodiments of the glucose sensor. In certain embodiments, the temperature sensing element can be a thermistor (as described above with regard to FIG. 1, and FIG. 4, reference numeral 25), a platinum resistance temperature device (“RTD”), another RTD, a thermocouple, an infrared-based temperature detector, a fluorescence-based temperature sensing element, or other temperature sensing elements with determinable temperature-dependent characteristics.

Devices such as thermistors, platinum RTDs, and other RTDs generally require one or more conductors, such as wires, to conduct the output of the sensor to a receiving unit which converts the output to a temperature signal. The conductors can be bundled with the optical fiber of fluorescence-based glucose sensors, such as those discussed above, or they can be routed separately. In one embodiment, the temperature sensor is placed inside the body, and the receiver is placed outside the body. In another embodiment, the temperature sensor is placed inside the body, and a transmitter, signal processor, etc. is also placed inside the body and is connected to or is a part of the temperature sensor. In preferred embodiments, the temperature sensing element is located at or near the glucose sensing moiety.

In another embodiment, a fluorescence-based temperature sensing technique can be used. Fluorescence-based temperature sensing techniques include those based on fluorescence decay, such as where an excitation light is provided to a phosphor, the excitation light is stopped, and the fluorescence is monitored versus time, with the rate of decrease in fluorescence being related to the temperature of the phosphor. Various techniques, can also include phase measurement and phase angle analysis.

Methods for performing fluorescence-based temperature measurement have been described. See for example, LumaSense Technologies, Inc. (Santa Clara, Calif.), “Fluoroptic Temperature Monitoring,” http ://www.lumasenseinc. com/technology/fluoroptic thermometry.html. Fluorescent materials that can be used in fluorescence-based temperature measurement are known to, or readily identified by those having skill in the art.

In some embodiments, the fluorescent material can be surrounded by material which prevents or inhibits chemical interaction between the fluorescent material and blood components. Suitable materials include glass (for example, borosilicate, lime-soda, or other types including those used for fiberoptic cables), polymers (for example, Teflon, fluoropolymers, silicone, latex, polyolefins, polyisoprene, and other rigid and nonrigid polymeric materials), metals (for example, 300 series stainless steel, 400 series stainless steel, nickel, nickel alloys, chromium steels, zirconium and its alloys, titanium and its alloys, as well as other corrosion resistant metals and alloys including exotic metals and alloys), ceramics (for example, ceramic materials related to aluminum oxide, silica and oxide, zirconium, carbides, etc.), and combinations of these.

In some embodiments, the temperature sensor can be positioned within the glucose sensor, or near it. While in one preferred embodiment, the temperature sensor can be positioned as close as possible to the glucose-sensing site(s) of the glucose sensor or made a part of the sensor, positions some distance away can also be successfully utilized, including those locations where the temperature measured provides an indication of the temperature at the glucose-sensing site(s) within an acceptable error for the use for which the temperature measurement is being made, such as for thermally compensating glucose readings or for detecting aberrant blood glucose readings.

In some embodiments, acceptable locations for the temperature sensor include those locations where an infusion of fluid having a different temperature from the blood upstream of the infusion point can be detected by the change in temperature of the fluid flowing through the blood vessel. Preferred locations include locations downstream of the infusion point and sufficiently close to the infusion point such that the fluid will not have been warmed to body temperature prior to contacting the temperature sensor.

In some embodiments, suitable locations can be less than about 2 mm up to about 100 mm or more upstream or downstream of the glucose sensor. In some embodiments, the two sensors can be contacting one another or be placed side-by-side in the bloodstream. The temperature stability of the portion of the body the glucose sensor is placed in can also affect the preferred proximity of the two sensors. For example, it can be preferable to place the temperature sensor closer to the glucose sensor when the glucose sensor is positioned in a portion of the body more subject to temperature fluctuation, such as the extremities, including when temperature fluctuations might be anticipated.

In some embodiments, the temperature sensor and/or the leads to the sensor can be isolated from the physiological environment, such as by coating, covering, or encasing the various parts with a material that prevents or inhibits chemical or physical interaction between the temperature sensor and/or its leads and blood components. Chemical interactions that are preferably avoided include corrosion, leaching of chemical species, generation of additional signals (e.g. optical, electrical, etc.) and take-up by the body of materials present in the sensor or leads, whether present from manufacture, corrosion or other means, such as compounds, metals, or ions causing a physiological response in some patients including copper, silver, organic compounds, organometallic compounds, etc.

Physical interactions can include breakage and physical separation (e.g. disconnection and potential loss), signal leakage (e.g. optical; electrical, etc.), signal degradation (including resistance, stray signal detection, noise, capacitance, electrochemical effects, induced voltages, ground loops, etc.). Suitable materials include glass (for example, borosilicate, lime-soda, as well as other types of glass, such as those used in production of optic fibers), polymers (for example, Teflon, fluoropolymers, silicone, latex, polyolefins, polyisoprene, acrylics, polycarbonates, and other rigid and nonrigid polymeric materials), metals (for example, 300 series stainless steel, 400 series stainless steel, nickel, nickel alloys, chromium steels, zirconium and its alloys, titanium and its alloys, as well as other corrosion resistant metals and alloys including exotic metals and alloys), ceramics (for example, ceramic materials related to aluminum oxide, silica and oxide, zirconium, carbides, etc.), and combinations of these.

Suitable methods for applying for isolating material to the temperature sensor or leads can include any appropriate method, including casting, painting, dipping, gluing, reacting, drawing, depositing, mechanically adhering, encapsulating, etc.

In some embodiments, small temperature sensors are preferred over large temperature sensors; although the relative size may vary depending on the desired configuration and placement site. Suitable sizes for temperature sensors that will be incorporated into the glucose sensor include those temperature sensing elements resulting in an overall glucose sensor of about 1 mm in diameter. However, in some embodiments, larger sizes can also be used such as when electrochemical glucose sensors are employed, when additional sensing features are included in the sensor assembly, when greater surface area or greater quantities of glucose-sensing material are desired, or when greater isolation from the physical environment is desired. However, frequently smaller diameter sensor assemblies are preferred, such as those having an outside diameter of about 600 microns, 400 microns, 300 microns, or smaller. Various sized leads, such as optical fibers and/or wires can be used. In some embodiments, an optical fiber having an outside diameter of about 500 micron, or preferably about 250 microns or about 200 microns or smaller can be used.

Wires, such as thermocouple wires or wires for resistance temperature devices, can be of a suitable size such as about 100 microns in diameter, or preferably about 50 microns or about 30 microns in diameter. The isolating material, in various embodiments can be adhered or fused, directly to the leads and/or sensor or made from the leads or sensor, such as through chemical reaction. However in some embodiments, a separate membrane can be provided as an isolating material that is tight-fitting, or loose-fitting as desired. For example, a membrane having an inside diameter of about 365 microns can be used to cover an optical fiber of about 250 microns into thermocouple wires of about 50 microns each. Generally, as the diameter of the leads and/or wires get smaller, the leads become more flexible, but can be more prone to breakage. In addition, signal transmission characteristics can also be affected as the diameter gets larger or smaller. Selection of the specific materials and the specific diameter can be determined by one of skill in the art considering such things as flexibility, strength, durability, electrical/optical losses, required length, etc.

### Combination of a Temperature and an Analyte Sensor

In some embodiments, a temperature sensor can be placed inside of, connected to, or made as a part of an analyte sensor. The temperature sensor can be positioned at various locations in reference to an analyte sensor, such as proximal, distal or alongside portions of the analyte sensor, or in a location that is a combination of distal, proximal and alongside parts of the analyte sensor. In some embodiments, the temperature sensor can be made of more than one part or portion and different portions can be positioned in different proximity to the analyte sensor.

In one embodiment, a temperature sensor can be located within an outer coating of the analyte sensor. Suitable outer coatings include polymers, glass, metal, elastomers, and ceramics. The coating can be porous, non-porous, or having a portion that is porous and a portion that is non porous, depending on such things as the proximity of the coating to the location of the portion of the sensor that functionally interacts with analyte in the environment.

In one embodiment, a temperature sensor can be affixed to the exterior of an analyte sensor, such as by adhesive, welding (metal, solvent, etc.), mechanical interaction, etc.

In one embodiment, a temperature sensor can be positioned inside an analyte sensor, such as within a sensor body, or within a gel matrix or polymeric matrix that makes up a functional, structural, or other part of an analyte sensor.

In various embodiments, a temperature sensor can be included in the analyte sensor during construction of the analyte sensor, or it can be added to the analyte sensor after the construction of the analyte sensor is substantially complete.

In some embodiments, an external sleeve or other enclosure can be added to the sensor to enclose or hold together the analyte sensor and temperature sensor.

In some embodiments, a hole or cavity can be created in the analyte sensor, such as during production of the analyte sensor or after, by molding, drilling, piercing, or other suitable method, and a temperature sensor inserted. Portions of the hole or cavity can be filled or covered, such as with adhesive, melted material, polymerizing material, solid material, sleeve, etc. Portions of the hole can be left uncovered or unfilled. In some embodiments, the entire hole or cavity remaining after addition of the temperature sensor can filled/covered or left unfilled/uncovered.

### Systems for Measuring in Vivo Analyte Concentration and Identifying Aberrant Measurements

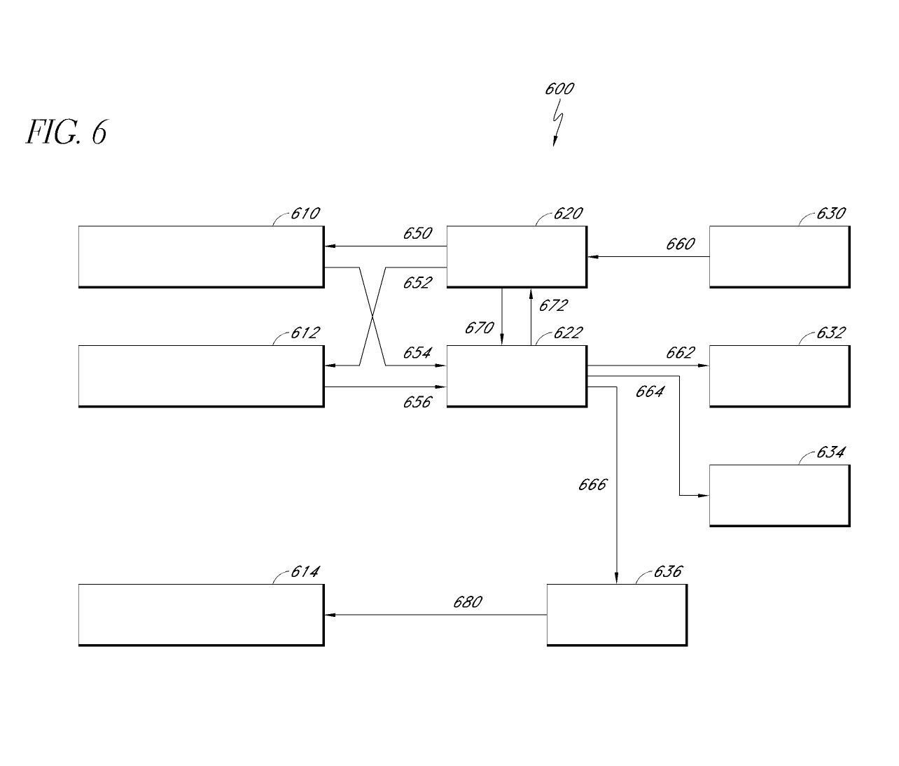

Disclosed herein are various systems for measuring an in vivo concentration of an analyte. For example, the block diagram in FIG. 6 schematically illustrates a system 600 for measuring an in vivo concentration of an analyte which comprise an analyte sensor 610, a temperature sensing element 612, at least one sensor control unit 620, and at least one receiving and processing unit 622.

In some embodiments, the analyte is glucose, and the analyte sensor 610 is a glucose sensor 610. Several suitable glucose sensors have been described in detail above. In some embodiments, the analyte sensor 610 is implantable and, in certain such embodiments, the analyte sensor 610 is configured for intravascular deployment (e.g., venous or arterial implantation) into a patient.

Suitable temperature sensing elements 612 have also been described above. Furthermore, as described above, a temperature sensing element 612 may be combined with an analyte sensor 610, or it may be a separate component. Thus, in certain embodiments, the temperature sensing element 612 may be configured for intravascular deployment along with the analyte sensor 610, such as, for example, a glucose sensor 610. Preferably, the temperature sensing element 612 is deployed intravascularly as closely as possible to the glucose-sensing site(s) of the glucose sensor 610, such that temperature readings received from the temperature sensing element 612 reflect the temperature at or near the glucose-sensing site(s) of the glucose sensor 610.

In addition to measuring an in vivo concentration of an analyte, some embodiments of the systems 600 identify whether the measurement is aberrant. There are numerous reasons why a measured analyte concentration may be aberrant. For instance, when the analyte is glucose, and the sensor 610 is a glucose sensor 610 implanted in a blood vessel, the glucose sensor 610 may be exposed to an aberrant condition at various times during use such as a “non-blood” environment or an environment where the blood is diluted with another fluid. Such an event can occur, for example, when fluids or medications are being administered intravenously. Similarly, an aberrant condition may also be created, for example, when the fluid being sensed by the glucose sensor is not the normal blood of the patient because whole or fractionated blood is being added to the patient. At these times, it is useful to identify the glucose measurement as aberrant, so that it may be treated appropriately. For example, to deal with a glucose measurement identified as aberrant by the system 600, a notation could be added to the glucose reading or an appropriate tag could be added to data representing glucose concentration. In some embodiments, the system 600 could even modify the output of a medication delivery device 614 such as a blood sugar controller or insulin pump to at least momentarily be insensitive to the glucose reading, as will be described in greater detail below.

Such conditions where the analyte sensor 610 is not measuring a representative condition of the patient can be detected, for example, by measuring temperature in the vicinity of the analyte sensor 610 with the temperature sensing element 612. For example, some embodiments of the system 600 detect an aberrant condition by measuring temperatures in the vicinity of the analyte sensor 610 with the temperature sensing element 612 at one or more times substantially close in time to the particular time the analyte concentration is measured by the analyte sensor 610, and also by measuring temperatures at one or more times previous in time to the particular time when the analyte concentration is measured. Comparison of the one or more temperatures measured contemporaneously with the analyte measurement, and the one or more temperatures measured prior to the analyte measurement may, in some embodiments, indicate whether or not a measured in vivo analyte concentration is aberrant. Thus, in some embodiments of the systems 600 schematically illustrated in FIG. 6, the analyte sensor 610 is configured to generate a signal indicative of the in vivo concentration of the analyte at a particular time, and the temperature sensing element 612 is configured to generate a plurality of signals which include at least one first temperature signal indicative of a temperature in the vicinity of the analyte sensor 610 previous in time to the particular time, and at least one second temperature signal indicative of a temperature in the vicinity of the analyte sensor 610 substantially close in time to the particular time. The specific details of how these temperature measurements are used in various embodiments of systems 600 to determine whether a measured analyte concentration should be identified as aberrant are described in greater detail below.

As schematically illustrated in FIG. 6 and mentioned above, a system 600 may include at least one sensor control unit 620. In some embodiments, the at least one sensor control unit 620 may be configured to control the operation of the analyte sensor 610 and the temperature sensing element 612. In certain embodiments schematically illustrated in FIG. 6, the sensor control unit 620 may be operably coupled to the analyte sensor 610 via signal path 650. Moreover, in certain embodiments schematically illustrated in FIG. 6, the sensor control unit 620 may be operably coupled to the temperature sensing element 612 via signal path 652. In some embodiments, the analyte sensor 610 and the temperature sensing element 612 are operably coupled to the sensor control unit 620 in such a way that the sensor control unit 620 may be placed, for example, on the patient's skin or clothing, on the bed, reversibly attached to the IV stand, on the bedside table of an ICU patient, or at the nurses' station. The coupling may be direct wire coupling, fiber optic coupling, transmission—receiver coupling (e.g., RF, IR, etc.), or any other art recognized component coupling means; in preferred embodiments, an optical fiber sensor is optically coupled to a sensor control unit.

Signal paths 650 and 652 in FIG. 6 schematically represent the operable couplings between the analyte sensor 610 and the sensor control unit 620, and between the temperature sensitive element 612 and the sensor control unit 620, respectively. However, the signal paths 650 and 652 should not be interpreted as implying that the aforementioned components are physically hard-wired together. As described above, the connection may be wireless. Furthermore, the aforementioned components may be formed integrally with one another such that the components, for example, the analyte sensor 610 and the sensor control unit 620, may be directly coupled together. Furthermore, in some embodiments, the signal path 650 between the sensor control unit 620 and the analyte sensor 610 may carry signals other than (or in addition to) control signals. For instance, in some embodiments, the signal path 650 may carry optical excitation radiation to an optical indicator system in the analyte sensor 610 (e.g., hydrogel immobilized fluorophore—glucose-binding quencher), as described with respect to FIGS. 1-5 above. In other embodiments, the signal path 650 may carry a voltage to be applied across the electrodes of an electrochemical sensor in the analyte sensor 610 (e.g., as disclosed in U.S. Pat. No. 6,565,509 and Patent Publ. Nos. 2008/0119704, 2008/0197024, 2008/0200788, 2008/0200789 and 2008/0200791; each of which is incorporated herein in its entirety by reference).

As schematically illustrated in FIG. 6 and mentioned above, a system 600 may include at least one receiving and processing unit 622. In some embodiments, the at least one receiving and processing unit 622 may be configured to receive various signals from the analyte sensor 610 via the signal path 654 schematically illustrated in FIG. 6. For instance, in some embodiments, the receiving and processing unit 622 may be configured to receive a signal indicative of the in vivo concentration of the analyte from the analyte sensor 610. Furthermore, in some embodiments, the at least one receiving and processing unit 622 may be configured to receive various signals from the temperature sensing element 612 via the signal path 656 schematically illustrated in FIG. 6. For instance, in some embodiments, the receiving and processing unit 622 may be configured to receive at least one first temperature signal indicative of a temperature in the vicinity of the analyte sensor 610 previous in time to the particular time at which the in vivo concentration was measured with the analyte sensor 610. In addition, in some embodiments, the receiving and processing unit 622 may be configured to receive at least one second temperature signal indicative of a temperature in the vicinity of the analyte sensor 610 substantially close in time to the particular time at which the in vivo concentration was measured with the analyte sensor 610.

In some embodiments, the analyte sensor 610 and the temperature sensing element 612 are operably coupled to the receiving and processing unit 622 (via signal paths 654, and 656, respectively) in such a way that the receiving and processing unit 622 may be placed, for example, on the patient's skin or clothing, on the bed, reversibly attached to the IV stand, on the bedside table of an ICU patient, or at the nurses' station—much the same as the sensor control unit 620. Again, the coupling may be direct wire coupling, fiber optic coupling, transmission—receiver coupling (e.g., RF, IR, etc.), or any other art recognized component coupling means. Thus, although the operable couplings between the aforementioned components are schematically illustrated by signal paths 654, and 656, these should not be interpreted as implying that the aforementioned components are physically hard-wired together. As described above, the connection may be wireless. Furthermore, the aforementioned components may be formed integrally with one another such that the components, for example, the analyte sensor 610 and the receiving and processing unit 622, may be directly coupled together. Moreover, in some preferred embodiments, the analyte sensor 610 is operably coupled to the receiving and processing unit 622 via an optical fiber which carries fluorescent emission from the optical indicator system in the analyte sensor 610 (e.g., hydrogel immobilized fluorophore—glucose-binding quencher) to the receiving and processing unit 622.

The sensor control unit 620 and the receiving and processing unit 622 may be operably coupled together via control paths 670 and 672 as schematically illustrated in FIG. 6. For instance, the sensor control unit 620 may adjust its operation of the sensors (the analyte sensor 610 and the temperature sensing element 612) based on signals it receives from the receiving and processing unit 622 over control path 670. In this manner, the sensor control unit 620 may be able to optimize the operation of the sensors 610, 612 based on their output as received by the receiving and processing unit 622. In other embodiments, it may be beneficial for the sensor control unit 620 to direct the operation of the receiving and processing unit 622 by sending signals over signal path 672. For instance, if the sensor control unit 620 is operating the sensors 610, 612 in a particular mode, the receiving and processing unit 622 may need to be set in a particular mode in order for the system 600 to operate properly.

Because the sensor control unit 620 and the receiving and processing unit 622 may be connected to the analyte sensor 610 and the temperature sensing element 612 in similar fashions, in some embodiments, the sensor control unit 620 and the receiving and processing unit 622 may be formed integral to one another, essentially, as a single unit. Forming these components integral with one another may facilitate operable coupling (e.g. signaling) between the components. However, the sensor control unit 620 and receiving and processing unit 622 may be distinct and still be operably coupled as illustrated schematically in FIG. 6 by the signal paths 670 and 672.

In some embodiments, the at least one receiving and processing unit 622 may be configured to identify whether a signal received from the analyte sensor 610 is indicative of an aberrant analyte concentration at the particular time the analyte concentration was measured. For instance, in some embodiments, the at least one receiving and processing unit 622 may generate a metric from the temperature signals received from the temperature sensitive element 612 over signal path 656. In certain such embodiments, the metric is generated from at least one first temperature signal that is indicative of a temperature (in the vicinity of the analyte sensor 610) previous in time to the particular time at which the analyte concentration was measured, and from at least one second temperature signal that is indicative of a temperature (in the vicinity of the analyte sensor 610) substantially close in time to the particular time at which the analyte concentration was measured. Thus, in certain embodiments, the metric generated is indicative of the difference in temperature in the vicinity of the analyte sensor between the particular time and one or more times previous in time to the particular time at which the analyte concentration was measured. It is actually possible to generate a variety of metrics from the aforementioned signals indicative of this temperature difference. Accordingly, several varieties of metrics are described below, as well as the details of how these various metrics are generated. For each metric, the receiving and processing unit 622 includes some sort of numeric processor capable of performing the relevant calculations.

The temperature difference (of which the various metrics are indicative of) may be significant because it may indicate the presence of an aberrant condition in the vicinity of the analyte sensor. For example, conditions in the vicinity of the analyte sensor may have changed drastically due to intravenous administration of fluids or medications as described above. An analyte concentration measured during such a period may not truly represent a patient's analyte level, because the patient's blood containing the analyte has been diluted. However, the fluid or medication administered intravenously is not likely to have the same temperature as the patient's blood. Thus, when the administered fluid mixes with the patient's blood, the temperature of the patient's blood in the vicinity of the mixing, or downstream from the mixing, may decrease. Since a decrease in a patient's blood temperature may roughly correspond to a dilution of the patient's blood, monitoring temperature in the vicinity of the analyte sensor 610 may be useful for identifying erroneous measurements of analyte concentration.

Accordingly, a metric generated from signals indicative of temperature in the vicinity of the analyte sensor as described above may be useful for predicting when a measured analyte concentration is aberrant. In certain embodiments, the receiving and processing unit 622 may generate the metric as described above and may compare the metric to a threshold value. Details of how the threshold value may be chosen/set are described below. For the case of a system 600 configured to measure intravascular glucose concentration, the threshold value may be set by the clinician, or it may be determined algorithmically based on previous temperature measurements, analyte measurements, or various calibration techniques. In certain embodiments, if the metric exceeds the threshold value, the receiving and processing unit 622 will identify the corresponding analyte concentration as aberrant.

Measurements of analyte concentrations taken after a prior concentration has been identified as aberrant may be handled in different ways depending on the embodiment. For example, in some embodiments, a system 600 can utilize a manual reset event, or it can utilize an automatic reset triggered by the return of normal conditions (e.g. temperature). Some embodiments may utilize a combination of a manual reset event and an automatic reset. Some embodiments of the system 600 may simply identify a measurement as aberrant and return to normal operation regardless of the conditions. In some embodiments employing an automatic reset, the receiving and processing unit 622 may make the determination whether the reset is called for. In some embodiments utilizing a manual reset event, a user would trigger the reset through the control panel 630 (described in greater detail below). Various examples of how a system 600 may employ an automatic reset triggered by the return of normal conditions after identifying a measurement as aberrant are described in detail below.

Some embodiments of the systems 600 may additionally include one or more of a control panel 630, a alert unit 632, a display unit 634, and a medication delivery device control system 636. Each of these devices are optional, yet each may provide added functionality when included in a system 600. Some combination of a control panel, an alert unit, and a display unit may collectively provide an interface for using the systems 600. For example, these components may provide the mechanism by which a clinical worker may operate the systems 600—e.g. when the systems 600 are used as glucose measuring devices. In other embodiments, a medication delivery device control system 636 may allow the system 600 to function in a fully or partially automated manner, so that the aforementioned components may require less attention from the clinical worker.

In some embodiments, the system 600 may comprise a control panel 630 which is operably coupled to the sensor control unit 620 via signal path 660 as schematically illustrated in FIG. 6. The control panel 630 may include of any sort of device that can receive manual input from a user such as a keyboard, a mouse, switches, jumpers, a scanner, a touch screen, or any other data entry device known in the art. The control panel 630 may also be adapted to accept data from another device such as a computer, a network of computers, a removable storage device, a chip, a barcode, a RFID, etc. Thus the control panel may include parallel ports, serial ports, USB ports, firewire ports, Ethernet ports, optical disc readers, magnetic disc readers, memory stick readers, Wi-Fi transmitters, barcode readers, RFID detectors, etc. Furthermore, although the control panel is only shown as operably coupled via signal path 660 to the sensor control unit 620, it may also be operably coupled to other components in some embodiments. For example, in some embodiments, the control panel is operably coupled to a display unit 634.

In embodiments comprising a display unit 634, the display unit may be operably coupled to the receiving and processing unit 622 via signal path 664. The display unit 634 may include any type of device capable of displaying information or data to a user of the system 600. For example, the display unit 634 may be a typical computer screen such as a CRT monitor, LCD monitor, plasma monitor, OLED monitor, or the like. In some embodiments, the display unit 634 may be simpler, such as just a row of LEDs which may indicate various conditions or states of the system 600. When the display unit 634 is capable of more complicated displays a wide variety of information and data can be shown. For example, a display unit 634 may display the signals indicative of analyte concentration or temperature as received from the receiving and processing unit 622 via signal path 664. In some embodiments, a display unit 634 may display values of analyte concentration or temperature, plots or graphs of these values versus time or versus other quantities such as heart rate. Generally, the display unit 634 may be used to display any value of clinical interest such as oxygen saturation level, heart rate, heart arrhythmia, levels of various blood analytes such as glucose, blood pressure (systolic and/or diastolic), etc. The display unit 634 may also display information or indicia indicating that a measured analyte concentration is aberrant as identified by the receiving and processing unit 622 over signal path 664.

Although not schematically indicated by a signal path in FIG. 6, in some embodiments, the display unit 634 may be operably coupled to the control panel 630 and/or the sensor control unit 620. For example, if the control panel 630 was configured to control certain aspects of the display, it might be operably coupled directly to the display unit 634 rather than have the control signals routed through the sensor control unit 620 and the receiving and processing unit 622. It should also be understood that FIG. 6 is not meant to imply that the various components—the control panel 630, the sensor control unit 620, the receiving and processing unit 622, and the display unit 634—must be physically distinct. For example, in one preferred embodiment of the system 600, each of these components comprise a general purpose computer which provides the requisite functionality to accomplish the tasks outlined above.

Some embodiments of the system 600 also include an alert unit 632. In some embodiments, such as the system 600 schematically illustrated in FIG. 6, the alert unit 632 is operably coupled to the receiving and processing unit 622 via signal path 662. In certain such embodiments, the alert unit 632 receives signals from the receiving and processing unit 622 via signal path 662 indicative of analyte concentration, and/or whether the analyte concentration is outside a prescribed range, and/or whether the analyte concentration has been identified as aberrant by the receiving and processing unit 622. After receiving such a signal, the alert unit generates an alert which may take the form of, for example, an audible alarm, a visual cue, or some other sensory stimulating alarm sufficient to alert a clinical worker of the relevant condition.

For example, if the system 600 comprises an intravascular glucose sensor, the alert could indicate to a clinical worker that a patient's blood glucose concentration is at or near a threshold level, or trending up or down at a threshold rate. For example, if blood glucose is monitored in an ICU patient, then an alarm may be used to alert the ICU staff that the patient is presently or soon to be hyper or hypoglycemic and requires intervention (e.g., administration of a glucose modulating agent) to maintain tight glycemic control. On the other hand, if the alert is indicative that a glucose reading produced by the system 600 is aberrant, then the alert would serve to warn ICU staff that the reading is suspect and should possibly be ignored when deterring whether to administer or withhold glucose treatment.

The alert unit 632 may also act to alert devices which may be in communication with the alert unit. For instance, any glucose monitors or displays may be used in accordance with aspects of the present invention. In addition, monitoring systems, such as patient monitoring systems or medical monitoring systems, including those systems having networking and computer functionality and the ability to monitor more than one patient, can be incorporated. In various embodiments, suitable monitoring systems can receive signals related to patient conditions and convert, store, display, record, transmit, etc. signals based on or derived from the received signals. Monitoring systems can be computer-based, or they can utilize other technology or combinations of technologies to provide the conversion, storage, etc. capability. In some embodiments, a monitoring system will have the display screen to display information related to the condition of the patient, such as medically relevant information, identification information, and/or other information including Doctor, contact information, etc. Of course much of this information could also be displayed on the display unit 634 as a component of the system 600, but in some circumstances a separate monitoring system, possibly monitoring multiple pieces of diagnostic equipment is appropriate. In some circumstances, use of a monitoring system possessing the capability of calculating relationships between parameters measured with multiple pieces of diagnostic equipment may be advantageous. In some circumstances, such information may include date, time of day, the time of meal, patient age, etc. Monitoring systems can also include data transmission or retransmission capabilities.

As discussed above, a patient's health is often dependent on the concentration and/or activity of certain analytes in the blood. Maintaining proper levels of bioavailability glucose, for example, has been found critical to the successful recovery of bedridden patients. Thus, monitoring and maintaining a patient's glucose activity (as well as the activity of other analytes) is an ongoing problem for hospital staff. Accordingly, systems 600 which can automatically (or somewhat automatically) regulate the in vivo concentrations and/or activities of analytes are extremely useful.

The system 600 may be operably coupled to a medication delivery device 614 via signal path 680 as schematically illustrated in FIG. 6. The medication delivery device 614 can be any device known in the art that is capable of delivering medication, fluids, analytes, etc. to the patient in response to an activating signal from another device. Accordingly, the system 600 may determine an analyte concentration in a patient as described above and operate the medication delivery device 614 by sending activating or deactivating signals to modulate a dosing regimen delivered to the patient by the medication delivery device 614. In some embodiments, the medication delivery device 614 may respond to the activating signal by delivering a fixed dose. In other embodiments, the medication delivery device 614 may respond to the activating signal by delivering fluids, analytes, medication, etc. until it receives a deactivating signal. In certain preferred embodiments, the medication delivery device 614 is an implantable device. In certain such embodiments, the medication delivery device 614 is configured for intravascular (arterial or venous) implantation so that it may deliver medication, analytes, fluids, etc. directly into the bloodstream. In some embodiments, the medication delivery device 614 is an intravascular glucose delivery device.

The system 600 may include, in some embodiments, a medication delivery device control system 636 to control the operation of the medication delivery device 614 as schematically illustrated in FIG. 6. The medication delivery device control system 636 may be operably coupled to the receiving and processing unit 622 via control path 666 and operably coupled to a medication delivery device 614 via control path 680. As described above, the receiving and processing unit 622 receives signals indicative of measured analyte concentration and identifies whether the measured concentrations are aberrant. The receiving and processing unit 622 may also have access to information relating to what ranges of analyte concentrations are biologically acceptable. For example, the receiving and processing unit 622 could be preprogrammed with biologically acceptable concentration ranges, or, for example, this information could be provided to the receiving and processing unit 622 through the control panel 630. In any case, in some embodiments, the receiving and processing unit 622 may be configured to monitor analyte concentration as measured by the analyte sensor 610, and compare the measured concentrations with a biologically acceptable predetermined range of analyte concentrations. In the event that the measured analyte concentration is less than the lower bound of the predetermined biologically acceptable range, the receiving and processing unit 622 may signal the medication delivery device control system 636 to operate the medication delivery device 614 to deliver analyte to the patient, or deliver a medication designed to trigger production of the analyte, or increase in vivo analyte concentration through some other mechanism). Similarly, in the event that the measured analyte concentration is higher than the upper bound of the predetermined biologically acceptable range, the receiving and processing unit 622 may signal the medication delivery device control system 636 to operate the medication delivery device 614 to deliver a medication designed to reduce production of the analyte or to decrease in vivo analyte concentration through some other mechanism. For instance, if the analyte of interest is glucose, then the system 600 may monitor the blood glucose levels of a patient, and maintain tight glycemic control by signaling the medication delivery device 614 to administering glucose in the event the patient is hypoglycemic and by signaling the medication delivery device 614 to administer insulin in the event the patient is hyperglycemic.

On the other hand, if the receiving and processing unit 622 has identified the measured analyte concentration as aberrant, the receiving and processing unit 622 may, in some embodiments, ignore the reading and not signal the medication delivery device control system 636 to operate the medication delivery device 614. The medication delivery device control system 636 could also respond to an aberrant reading by operating the medication delivery device 614, but instructing it to respond in an attenuated fashion. For example, if the reading is very low which would normally prompt the medication delivery device control system 636 to deliver a large dose, the medication delivery device control system 636 could respond to a very low reading identified as aberrant by signaling a moderate dose. In embodiments, where the medication delivery device 614 continues a steady-state operation until it receives a different signal from the medication delivery device control system 636, the medication delivery device control system 636 could respond to an aberrant reading by either sending a signal directing the medication delivery device 614 to stop delivery of medication, or simply ignore the reading, send no signal, and allow the medication delivery device 614 to continue its previous dosing regimen.

In some embodiments, the system has an algorithm or program that allows the various modules and components to operate in conjunction. In one embodiment, the system or monitor comprises a processor configured to execute certain algorithms or operational programs/processes. In one embodiment, the monitor measures the temperature of the subject and stores the information into a memory module. The monitor continues to measure the temperature of the subject over time and continues to store the temperature information associated with the measurement time information into the memory module. In some embodiments, the memory module also stores other information that is acquired by the monitor or operably coupled systems. The system can also comprise a sensor control unit that can control the operation of the glucose sensor and the temperature sensor. For example, the sensor control unit has a processor or is in communication with such a processor and measures and communicates the glucose and temperature information to the system.

In some embodiments, the system also has one or more receiving and processing unit. In one embodiment, the receiving and processing unit receives the temperature and glucose information from the sensor or sensor control unit. The sensor or sensor control unit can have a memory device to store the information as it is acquired or use a memory device somewhere in communication with the receiving and processing unit. In one embodiment, the receiving and processing unit has a processor and algorithm to track and compare the temperature measurement as it is acquired over time. In certain embodiments, the receiving and processing unit can analyze the collected and stored data either in its raw signal, or after it has been calculated to a user readable form, such as ° C.

In some embodiments, a processor is configured to control the operation of receiving the temperature signal and associated time information, and compare it to a temperature signal and associated time information received at a separate time. In one embodiment, the processor receives the temperature signal at the time of an in-vivo calibration and sets that temperature as the reference point for all later temperature measurements as the system is put into use. In one embodiment, the processor receives the temperature signal and compares it the signal of the immediately preceding signal. In one embodiment, the processor compares the temperature signal to a temperature signal not immediately preceeding, but more than one signals prior.

In some embodiments, the processor is configured to compare one or more temperature signals acquired over a length of time to determine the magnitude. In one embodiment, the processor receives temperature signal of multiple time points and compares them to each other continuously, to determine whether the temperature measurement has gone over the threshold at any point during the use of the system. For example, the difference in temperature may be determined, followed by dividing the difference by the time differential to determine the relative rate of change in the form of ° C./min. The processor may conduct additional data processing, such as taking derivatives of the data, for example to output a ° C./min².

In some embodiments, the processor triggers an alarm or alert the user or system there may be an aberrant glucose measurement, when the magnitude or rate of change is beyond a predetermined threshold. For example, if the temperature has changed according the one of the processes described above and exceeds the threshold, for example 0.4° C./minutes, the processor will trigger the alarm or alert the system. If it does not exceed the threshold, the system continues to operate. The processor can also be configured to generate a metric from the temperature signal indicative of an earlier temperature signal, which is indicative of the difference in temperature at the glucose sensor between a first time and a second time. The processor can be configured to identify the glucose concentration as aberrant if the metric exceeds a threshold value and output the information, for example to a display or external glucose controlling device, or an alarm.

### Methods of Identifying Aberrant Measurements of Analyte Concentration

Also disclosed herein are various methods of identifying whether a measured analyte concentration is aberrant. In some embodiments, a method of identifying whether a measured analyte concentration—measured by an in vivo analyte sensor at a particular time—is aberrant comprises generating at least one first temperature signal and at least one second temperature signal. In some embodiments, the at least one first temperature signal is indicative of a temperature in the vicinity of the analyte sensor previous in time to the particular time. In some embodiments, the at least one second temperature signal is indicative of a temperature in the vicinity of the analyte sensor substantially close in time to the particular time. In some embodiments, various methods comprise generating a metric from the at least one first temperature signal and the at least one second temperature signal. In some embodiments, the metric is indicative of the difference in temperature in the vicinity of the analyte sensor between the particular time the analyte concentration was measured and one or more times previous in time to the particular time the analyte concentration was measured. Various metrics that fit this criteria may be generated in accord with certain embodiments described herein. Some of these metrics and how they are generated are described in greater detail below. In some embodiments, an aberrant analyte concentration is identified if the metric corresponding to the analyte concentration measurement exceeds a threshold value. In some embodiments, the method further comprises alerting a user, alerting a monitor, and/or signaling a medication delivery device when an aberrant analyte measurement is identified.

Also disclosed herein are various methods of administering medication to a patient in response to a measured concentration of an analyte in the patient. In some embodiments, the methods may comprise providing an analyte sensor to the patient in vivo, and a temperature sensing element in the vicinity of the analyte sensor. The methods may further comprise measuring the concentration of the analyte using the analyte sensor, and identifying whether the measured concentration of the analyte is aberrant using the temperature sensor according to any of the methods disclosed in the previous paragraph. In certain such embodiments, medication is administered to the patient in response to the measured concentration of the analyte if and only if the measured concentration is not aberrant.

### Metrics and Threshold Values for Detection of Aberrant Conditions

As described above, systems and methods disclosed herein for measuring in vivo analyte concentration use various metrics and threshold values for the detection of aberrant measurements of analyte concentration. Also, in some embodiments, as described above, these metrics correspond to an analyte concentration measured at a particular time, and they are may be indicative of the difference in temperature in the vicinity of the analyte sensor between the particular time the analyte concentration was measured and one or more times previous in time to that particular time. As such, the metric is oftentimes generated from at least one first temperature signal that is indicative of a temperature (in the vicinity of the analyte sensor) previous in time to the particular time at which the analyte concentration was measured, and from at least one second temperature signal that is indicative of a temperature (in the vicinity of the analyte sensor 610) substantially close in time to the particular time at which the analyte concentration was measured.

However, within this general framework, a variety of metrics may be used as indicators of aberrant conditions. For instance, in some embodiments where the at least one first signal is just a single temperature reading taken prior to the measurement of analyte concentration, and the at least one second signal is just a single temperature reading taken nearly simultaneously with the corresponding measurement of analyte concentration, the metric could simply be the magnitude of the difference between these two temperatures. Once the value of the metric is determined as such it may be compared to a predetermined threshold value. Typically, if the value of the metric does not exceed the threshold value, then the analyte concentration measurement will not be identified as aberrant. Conversely if the metric does exceed the threshold value, then the analyte concentration will be identified as aberrant. In some embodiments, suitable threshold values may include, but are not limited to, 0.1° C., 0.2° C., 0.3° C., or 0.4° C. Thus, for example, if the threshold value is set to 0.2° C., the temperature indicated by the first signal is 37.0° C., and the temperature indicated by the second signal is 36.5° C., then the corresponding analyte concentration measurement would be identified as aberrant. As described above, temperature differences may be effective at identifying aberrant measurements of analyte concentration because temperature differences may indicate the presence of an aberrant condition present in the region that the analyte concentration was measured. For example, if analyte concentration is being measured intravenously, then temperature differences over time may be effective at indicating, for example, that there has been an intravenous administration of non-bodily fluids, since the non-bodily fluid likely has a different temperature than the patient's blood (as discussed above). In other embodiments, a metric which may be generated and compared to a threshold is the magnitude of the difference between a standard temperature and the temperature reading taken nearly simultaneously with the corresponding measurement of analyte concentration.

In other embodiments, the absolute difference in temperature may be more significant than the magnitude of the difference. For example, if the most significant aberrant condition throwing off analyte measurements is the intravenous administration of non-bodily fluids, and the non-bodily fluids typically have a temperature less than that of a patient's blood, then decreases in blood temperature are a more likely indicator of aberrant conditions than increases in blood temperature. Accordingly, in some embodiments, the metric is the actual difference in temperature (positive or negative, rather than the magnitude of the change) and the threshold value might be a negative temperature value. In other words, the temperature has to decrease by more than the magnitude of the threshold value before the corresponding measured analyte concentration is identified as aberrant. In other embodiments, for example where a positive change in temperature is a likely indicator of an aberrant condition, the actual difference in temperature may be used as the metric and the threshold value may be set to a positive temperature.

However, in some embodiments, more complicated metrics may be used. For instance, if either the at least one first signal indicative of temperature or the at least one second signal indicative of temperature include more than one signal and are indicative of more than one temperature, then the metric may be the magnitude of the difference between an average of temperatures indicated by the at least one first temperature signal and an average of temperatures indicated by the at least one second temperature signal. In some embodiments, the metric may be the difference between a temperature measured nearly simultaneously with the analyte measurement and the median of all past temperature measurements. In some embodiments, the metric may be the magnitude of the difference between the median temperature indicated by the at least one first signal indicative of temperature and the median temperature indicated by the at least one second signal indicative of temperature.

In some embodiments, the metric is derived from a model of temperature as a function of time based on all the measured temperatures (e.g. all the temperatures indicated by the first signal and the second signal) or a subset thereof. The model could be, for example, a polynomial function fit to the temperatures using a least squares fit. The metric could be chosen to be a quantity indicative of erratic temperature fluctuations close in time to the particular time the analyte concentration is measured. As before, if the erratic temperature fluctuations quantified by the metric exceed some threshold, the corresponding analyte concentration could be identified as aberrant. In certain such embodiments, the metric could be a first or second derivate of temperature with respect to time generated from the polynomial fit and evaluated close in time to the particular time the analyte concentration was measured.

In some embodiments, the metric is derived from a statistical model based on all the measured temperatures (e.g. all the temperatures indicated by he first signal and the second signal) or a subset thereof, which may be used to estimate the probability that a temperature jump at a particular time is due to a random fluctuation or due to a non-random (systematic) change in the environment local to measurement of analyte concentration. The threshold value would be chosen as some cutoff probability that the fluctuation is non-random. For example, if the statistical model predicted that a temperature change/jump contemporaneous with the analyte measurement is more likely to be non-random than the cutoff value, the analyte concentration measurement would be identified as aberrant. Various parameters that can be utilized in such an analysis can include values for variants, standard deviation, mean, etc.

In some embodiments, the metric is derived from one or more first or second derivates of temperature (or signals indicative of temperature) with respect to time. First and second derivates may be estimated by any method known in the art including, but not limited to, various finite differencing schemes, or by fitting a polynomial to the data and evaluating the derivative of the polynomial analytically. In some embodiments, only two data points are used to estimate/compute a first derivate of temperature versus time, while in other embodiments, more than two data points may be used. In some embodiments, only three data points are used to compute a second derivative with respect to time, while in other embodiments more than three data points may be used.

In some embodiments, the metric is the magnitude of a first derivative of temperature (or a signal indicative of temperature) with respect to time evaluated at substantially near the particular time the analyte concentration was measured. In certain such embodiments the threshold value for comparison is set at 0.1° C./min., 0.2° C./min., 0.3° C./min., or 0.4° C./min. In some embodiments, the metric is the magnitude of the difference between a first derivative of temperature (or a signal indicative of temperature) with respect to time evaluated at substantially near the particular time the analyte concentration was measured and a first derivative of temperature (or a signal indicative of temperature) with respect to time evaluated at a previous time. In certain such embodiments the threshold value for comparison is set at 0.1° C./min., 0.2° C./min., 0.3° C./min., or 0.4° C./min.

In some embodiments, the metric is the magnitude of a second derivative of temperature (or a signal indicative of temperature) with respect to time evaluated at substantially near the particular time the analyte concentration was measured. In certain such embodiments the threshold value for comparison is set at 0.1° C./min,², 0.2° C./min.², 0.3° C./min.², or 0.4° C./min.². In some embodiments, the metric is the magnitude of the difference between a second derivative of temperature (or a signal indicative of temperature) with respect to time evaluated at substantially near the particular time the analyte concentration was measured and a second derivative of temperature (or a signal indicative of temperature) with respect to time evaluated at a previous time. In certain such embodiments the threshold

The relationship between actual temperature and signals indicative of temperature should be understood throughout the preceding discussion. Signals indicative of temperature are simply signals that bear some relationship to temperature. Generally, a device which generates a signal indicative of temperature may or may not use that signal to compute an actual temperature. However, computing an actual temperature may not be necessary. Oftentimes, useful information can be obtained from deriving metrics and thresholds from signals indicative of temperature rather than actual temperatures themselves. As such, it is to be understood that when temperature is discussed in the context of this disclosure, the term encompasses related quantities (such as signals indicative of temperature) which would allow the proper functioning of the systems disclosed herein and the proper use of the methods disclosed herein. In the particular context of describing the metrics and thresholds used in the systems and methods disclosed herein, referring to temperature is often more convenient. However, such use should be read to limit the scope of this disclosure.

Reset after Detection of Aberrant Conditions

As described above, in some embodiments, a system 600 may require a manual reset event or an automatic reset before returning to normal operation after an analyte measurement has been identified as aberrant. Similarly, some methods of administering medication to a patient in response to a concentration of an analyte in the patient, may require a return to normal conditions (e.g. normal temperatures) after identifying a certain measured concentration of analyte as aberrant before administration of the medication may be resumed.

In some embodiments, automatic reset may be triggered by consideration of the temperature, changes in temperature, the rate of change in the temperature (e.g. the first and second derivatives of temperature with respect to time), the analyte concentration, or a combination of these parameters. In one embodiment, automatic reset may be triggered by a return of the temperature to a pre-aberrant value, or to within a preset range of a pre-aberrant value. In one embodiment, automatic reset may be triggered by a reversal of the rate of change in the temperature, such as a positive value for the rate of change following an aberration condition identified with a negative rate of change of the temperature, or vice versa. In some embodiments, a reversal of the rate of change in the temperature can be combined with the identification of an asymptote in the temperature or a reversal in the rate of change followed by a decrease in the absolute value of the rate of change of temperature. In some embodiments, evaluation of the analyte readings can also be utilized, such as by identifying an asymptote the concentration following a decrease in concentration followed by an increase in concentration. In some embodiments, automatic reset may be triggered by identifying or noting when the infusion of fluids is complete.

### Additional Methods, Techniques, and Systems for Alerting Medical Staff to Potential Aberrant Glucose Readings

In various embodiments, methods, techniques, and systems can be implemented to prevent an aberrant glucose reading from being used in the treatment of a patient. Suitable methods, techniques, and systems can include procedures, including those performed by medical staff, such as inspection of a display or a record for an indication of an aberrant reading. Suitable methods, techniques, and systems can also include display of an audible or visual signal indicating that an aberrant reading is present or suspected. Such signals can be presented on a display screen or other visual signal system locally or remotely, such as in a local medical monitor, or in a centralized monitoring system, sent to a beeper, computer, pager, telephone etc., enunciated locally or remotely, or otherwise indicated to appropriate personnel to determine appropriate action.

In some embodiments, an indication of aberrant condition can be made in conjunction with the display of a measured parameter. Suitable indications include, but are not limited to a notation on the display screen, a change in the color or other visual characteristics of a measured parameter on a display screen, substitution of a different value for the measured value, such as a value indicated of an aberrant reading or an estimate of the reading that would occur in the absence of an aberrant condition, or other suitable indication.

In some embodiments, an aberrant condition would require acknowledgment by appropriate personnel. In some embodiments, an aberrant condition signal could be cleared automatically upon resolution of the aberrant condition or resolution of conditions interpreted as representing an aberrant condition.

Additional methods, techniques, and systems can include those which record the occurrence of an aberrant condition. Such recording can be done in a medical file, a record of the parameters being monitored, etc. In some embodiments, the presence of an aberrant condition can be noted in proximity to the parameter suspected of being aberrant or it can be noted in place of or along with the suspected parameter. Such recording can be done on paper, on computer readable materials, or elsewhere as appropriate to the situation.

Analyte Sensor Temperature Correction

An analyte sensor may exhibit a temperature dependence such that signals generated by the analyte sensor may vary with temperature even if the underlying concentration being measured is constant. For instance, in the case of a glucose sensor, the output signal of the sensor depends on the temperature of the sensor and the sensing environment. This temperature sensitivity is true for fluorophore-quencher-based sensors, such as those described in U.S. patent application Ser. No. 11/671,880 [GLUM.004A], incorporated herein by reference in its entirety, as well as other sensing techniques. Due to the different phenomena involved in how different types of sensors work, a different relationship between temperature and reading or temperature and adjustment may be required for different analyte sensors.

Thus, in addition to using temperature to identify aberrant analyte concentration measurements, some embodiments of systems and methods disclosed herein may use measurements of temperature in the vicinity of an analyte sensor to correct for the temperature dependence of the analyte sensor.

In some embodiments, a relationship can be developed usable for temperature compensation/adjustment by determining analyte readings at different temperatures, such as over a biologically significant temperature range, or a range that might be encountered in the bloodstream of a subject. The temperature range can include temperatures that can be encountered under aberrant conditions, such as when a fluid infusion is performed, or under conditions encountered in environmentally or physiologically extreme conditions, such as in extreme cold, extreme heat, intense exercise, thermal treatment of a patient, etc.

In various embodiments, data relating temperature to readings or adjustment of readings can be utilized as a correlation. Suitable correlations include utilizing the data or information related to or derived from the data in a look-up table or in another form, including a statistical or mathematical correlation. Suitable statistical and mathematical correlations can include least squares analysis, multivariable least squares, linear correlations, nonlinear correlations, transformation of variables, as well as other techniques for correlating or relating data to other parameters.

In various embodiments, a relationship between temperature and analyte concentration/correction/adjustment can be implemented in a computer system. Suitable computer systems include those which can read a signal related to temperature and a signal related to analyte concentration, and output a correction or adjustment to the analyte signal, or a signal or reading related to a corrected analyte concentration. Suitable computer systems can utilize look-up tables or other techniques and correlations such as mathematical relationships for interpolating data or correlating data by other techniques. In some embodiments of the system 600 schematically illustrated in FIG. 6, the receiving and processing unit 622 may compute temperature corrected analyte concentrations based on signals it receives from the temperature sensing element 612.

### EXAMPLE 1

A sensor comprising a glucose sensor and a temperature sensor made according to the present disclosure is prepared. The sensor is calibrated by using at least one calibration solution with a known glucose concentration. The sensor is deployed intravascularly into the subject and allowed to stabilize. The proximal end of the sensor is coupled to a light source and a programmable monitor adapted to display continuous real-time glucose measurements as well as rates and directions of changes in blood glucose levels. The monitor is programmed to generate an alarm when it detects an aberrant glucose measurement based on a threshold set to 0.4° C./min.

The sensor and monitor are put into operation mode where the light source emits light at a fixed frequency of every 15 seconds. The glucose sensor is excited by the light and emits an emission that is indicative of the glucose activity. The glucose sensor measures a glucose activity, which is optionally converted to a glucose concentration measurement. A sample blood is drawn from the subject and measured with a lab analyzer to acquire an independent glucose measurement. A one-point in vivo calibration is done wherein the independent glucose measurement is input into the monitor to adjust the monitor readings from the glucose sensor to correspond to the independent glucose measurement.

At a first time, the glucose sensor measures the glucose concentration is about 100 mg/dL and the temperature sensor is measuring 37.0° C. At the same first time, IV fluid is infused into the subject upstream of the same vascular line used for the glucose measurements. At a second time about 15 seconds after the infusion, the sensor measures the glucose concentration to be about 102 mg/dL at 36.9° C. At a third time about 30 seconds from the infusion, the sensor measures the glucose concentration to be about 104 mg/dL at 36.8° C. At a fourth time about 45 seconds from the infusion, the sensor measures the glucose concentration to be about 120 mg/dL and the temperature sensor is measuring 36.5° C. The monitor detects that the temperature measured at the third time and fourth time is different by more than the threshold amount of 0.4° C./min and triggers the alarm. The alarm alerts the medical staff that the glucose measurement is potentially aberrant because of the IV fluid infusion and allows the medical staff to make appropriate decisions for further medical treatment. Instead of immediately administering insulin to the patient, the medical staff monitors the patient for several more glucose and temperature readings to allow the readings to stabilize.

### EXAMPLE 2

A sensor is inserted substantially similarly to Example 1, except the alarm is set to trigger when the average temperature of the first and third times is different by more than 0.4° C. than the average temperature of the second and fourth times. At a first time, the glucose level is about 100 mg/dL and the temperature sensor is measuring 37.0° C. At the same first time, IV fluid is infused into the subject upstream of the same vascular line used for the glucose measurements. At a second time about 15 seconds after the infusion, the sensor measures the glucose concentration to be about 95 mg/dL at 36.9° C. At a third time about 30 seconds from the infusion, the sensor measures the glucose concentration to be about 95 mg/dL at 36.8° C. At a fourth time about 45 seconds from the infusion, the sensor measures the glucose concentration to be about 90 mg/dL and the temperature sensor is measuring 36.9° C. At a fifth time about 60 seconds from the infusion, the sensor measures the glucose concentration to be about 75 mg/dL and the temperature sensor is measuring 36.6° C. The monitor detects that the average temperature of the second and fourth time (36.9° C.) is different by more than the threshold amount of 0.4° C./min than the average temperature of the third and fifth time (36.7° C.) and triggers the alarm. The alarm alerts the medical staff that the glucose measurement is potentially aberrant because of the IV fluid infusion. Instead of immediately administering insulin to the patient, the medical staff monitors the patient for several more glucose and temperature readings to allow the readings to stabilize. Continuous readout of the rate and direction of blood glucose trend and monitoring for aberrant measurements allows the medical staff to determine whether intervention is needed. The ICU staff is able to maintain control of the patient's blood glucose concentration and the recovery is smooth and no critical illness polyneuropathy or other complications are observed.

This invention may be embodied in other specific forms without departing from the essential characteristics as described herein. The embodiments described above are to be considered in all respects as illustrative only and not restrictive in any manner. The scope of the invention is indicated by the following claims rather than by the foregoing description. Any and all changes which come within the meaning and range of equivalency of the claims are to be considered within their scope.

What is claimed is:

1. A method of identifying whether an intravascular glucose measurement is aberrant, the method comprising:
deploying a sensor within a blood vessel, wherein the sensor comprises along a distal region thereof, a temperature-sensing element adapted to measure the temperature of the blood contacting the distal region, and a glucose-sensing chemical indicator system adapted to measure the glucose concentration in the blood contacting the distal region;
generating at a first time, a first temperature measurement (TO;
generating at a second time, a second temperature measurement (T₂) and a glucose measurement;
comparing T₁ and T₂; and
identifying the glucose measurement as aberrant if the absolute magnitude of the difference between T₁ and T₂ exceeds a pre-selected threshold value.

2. The method of claim 1, further comprising alerting a user, alerting a monitor, and/or signaling a medication delivery device when an aberrant glucose measurement is identified.

3. The method of claim 1, wherein the threshold value is 0.1° C.

4. The method of claim 1, wherein the threshold value is 0.2° C.

5. The method of claim 1, wherein the threshold value is 0.3° C.

6. The method of claim 1, wherein the threshold value is 0.4° C.

7. A method of identifying whether an intravascular glucose measurement is aberrant at a given time, the method comprising:
deploying a sensor within a blood vessel, wherein the sensor comprises along a distal region thereof, a temperature-sensing element adapted to measure the temperature of the blood contacting the distal region, and a glucose-sensing chemical indicator system adapted to measure the glucose concentration in the blood contacting the distal region;
generating one or more temperature measurements prior to the given time;
generating simultaneously, at the given time, a temperature measurement and a glucose measurement;
comparing the temperature measurement generated at the given time with an average of the temperature measurements generated prior to the given time; and
identifying the glucose measurement generated at the given time as aberrant if the absolute magnitude of the difference between the temperature measurement generated at the given time and the average of the temperature measurements generated prior to the given time exceeds a pre-selected threshold value.

8. A system for measuring an in vivo glucose concentration and identifying whether the measurement is aberrant, the system comprising:
a glucose sensor comprising a chemical indicator system configured to generate a signal indicative of the in vivo concentration of the glucose at a first time;
a temperature sensing element configured to generate a plurality of signals comprising:
a first signal indicative of temperature at a second time prior to the first time; and
a second signal indicative of temperature at the first time;
at least one sensor control unit, the at least one sensor control unit configured to control the operation of the glucose sensor and the temperature sensing element; and
at least one receiving and processing unit, the at least one receiving and processing unit configured to:
receive the signal indicative of the in vivo concentration of the glucose;
receive the first signal indicative of temperature;
receive the second signal indicative of temperature;
generate a metric from the first signal indicative of temperature and the second signal indicative of temperature, the metric indicative of the difference in temperature at the glucose sensor between the first time and second time; and
identify the glucose concentration as aberrant if the metric exceeds a threshold value.

9. The system of claim 8, wherein the metric is the magnitude of the difference between the first signal indicative of temperature and the second signal indicative of temperature divided by the elapsed time between generation of the signals.

10. The system of claim 8, further comprising an alert unit, the alert unit generating an alert when a measured concentration of the glucose is identified as aberrant.

11. The system of claim 8, further comprising a display unit, the display unit configured to indicate the measured concentration of the glucose.

12. The system of claim 11, wherein the display unit is further configured to indicate when a measured concentration of the glucose is identified as aberrant.

13. The system of claim 8, further comprising a medication delivery device control system, the medication delivery device control system configured to generate signals adapted to control a medication delivery device, the signals comprising:
a signal indicative of a measured in vivo glucose concentration; and
a signal indicative of whether the measured in vivo glucose concentration has been identified as aberrant.

14. The system of claim 13, wherein the threshold value is between 0.1° C. and 0.4° C.
